(19) United States
(12) Patent Application Publication
(10) Pub. No.: US 2009/0181412 A1
(43) Pub. Date: Jul. 16, 2009

(54) METHOD OF STORING ANALYTICAL REAGENT INTO MICROFLUIDIC DEVICE

(75) Inventors: Yoon-kyoung CHO, Suwon-si (KR); Hee-Kyun LIM, Suwon-si (KR); Jeong-gun LEE, Seoul (KR); Ki-eun KIM, Seoul (KR); Sung-ho LEE, Anyang-si (KR); Chae-joon LEE, Anyang-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD., Suwon-si (KR)

(21) Appl. No.: 12/332,446

(22) Filed: Dec. 11, 2008

(30) Foreign Application Priority Data
Jan. 16, 2008 (KR) 10-2008-0004951

(51) Int. Cl. C12Q 1/62; C12Q 1/48; C12Q 1/32; C12Q 1/42; G01N 33/92; G01N 33/00; C12Q 1/60; C12Q 1/58; C12Q 1/54

(52) U.S. Cl. 435/10; 435/15; 435/26; 435/21; 435/11; 435/12; 435/14; 436/71; 436/86

(57) ABSTRACT

Provided is a method of storing a reagent in a microfluidic device. The reagent, in a liquid form, is loaded into reaction chambers arranged on the microfluidic device; followed by lyophilization as being contained in the microfluidic device.

### CROSS-REFERENCE TO RELATED PATENT APPLICATION

This application claims the benefit of Korean Patent Application No. 10-2008-0004951, filed on Jan. 16, 2008, in the Korean Intellectual Property Office, the disclosure of which is incorporated herein in its entirety by reference.

### BACKGROUND OF THE INVENTION

1. Field of the Invention

The present invention relates to a method of storing a analytical reagent, into a microfluidic device, and more particularly, to a method of storing a reagent into a microfluidic device for performing various analysis of a biological sample.

2. Description of the Related Art

Conventional pathological blood tests require various hand-held works and equipments. In order to rapidly perform the test, skilled clinical pathologists are needed. However, performing various tests at the same time is difficult, even for the skilled clinical pathologists. In diagnosing emergency patients, quick test results are important for timely emergency treatments. Accordingly, equipments enabling simultaneously, rapidly and accurately performing various pathological tests, required according to circumstances, are needed.

In case of conventional blood tests, large and expensive automated equipments are used and a relatively large amount of blood is required. Moreover, some of the tests take several days or weeks to obtain results. Smaller and automated devices, such as a microfluidic device, which enable rapid analysis of blood have been proposed.

In a rotatable disc-type microfluidic device, blood sample is separated into serum an plasma by centrifugal force generated by the rotation of the device. The separated serum is mixed with a diluent and the mixture is moved to a plurality of reaction chambers in the device. The reaction chambers each contain pre-loaded reagents employed for different tests of the serum. Reactions between the serum and the reagents may be detected using various signals, for example an optical signal, which are generated by reaction products.

For such a microfluidic device, it is difficult to store pre-loaded reagents in liquid state in the device. U.S. Pat. No. 5,776,563 discloses that various kinds of reagents are formulated respectively into a lyophilized bead form and introduced to reaction chambers of the microfluidic device, when a blood analysis is performed using the device.

### SUMMARY OF THE INVENTION

The present invention provides a method of storing reagents to a microfluidic device for a blood chemistry analysis, the reagents being loaded and stored by a fixed quantity in the microfluidic device to automatically perform the biochemical or pathological analysis of a biological sample.

According to an aspect of the present invention, there is provided a method of storing a reagent in a microfluidic device, wherein the reagent is used for assay of a biological sample and the microfluidic device is provided with a reaction chamber and fluid paths, the method including: loading the reagent into the reaction chamber; and lyophilizing the reagent in the state that the reagent is loaded into the microfluidic device.

In loading the reagent, the reagent may be a liquid reagent.

The reagent may be concentrated to a concentration that is higher than the concentration used in a test and may be loaded into the reaction chambers.

The lyophilizing may include a freezing process and a drying process, the drying process using sublimation in at least some portions of the drying process.

The microfluidic device may include at least two reaction chambers and the loading the reagent may include loading a plurality of the reagents that are different from each other into at least two reaction chambers.

The plurality of the reagents may include at least two reagents used for testing aspartate aminotransferase (AST), alanine aminotransferase (ALT), gamma glutamyl transferase (GGT), direct bilirubin (D-BIL), total bilirubin (T-BIL), creatin kinase (CK), Lactate Dehydrogenase (LDH), amylase (AMY), Creatinine (CREA), Albumin (ALB), Total Protein (TP), calcium (Ca), Urea Nitrogen (BUN), Alkaline Phosphatase (ALP), glucose (GLU), total cholesterol (CHOL), triglycerides (TRIG), and Uric acid (UA).

The method may further include adding a filler into the reagent. The filler may include at least one of bovine serum albumin (BSA), polyethylene glycol (PEG), dextran, mannitol, polyalcohol, myo-inositol, and citric acid.

The method may further include adding a surfactant into the reagent before lyophilizing the reagent. The surfactant may include at least one selected from the group consisting of polyoxyethylene, lauryl ether, octoxynol, polyethylene alkyl alcohol, nonylphenol polyethylene glycol ether; ethylene oxid, ethoxylated tridecyl alcohol, polyoxyethylene nonylphenyl ether phosphate sodium salt, and sodium dodecyl sulfate.

According to another embodiment of the invention, there is provided a method for producing a microfluidic device suitable for an assay of a biological sample, the microfluidic device is provided with a reaction chamber and fluid paths and contains a reagent for performing the assay in the reaction chamber, the method comprising: loading the reagent in liquid form into the reaction chamber of the device; and lyophilizing the reagent in the state that the reagent is loaded into the microfluidic device.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram of a microfluidic device for a blood chemistry analysis to which a method of storing reagent is applied, according to an embodiment of the present invention;

FIG. 2 is a schematic diagram of a blood analyzer using a microfluidic device; and

FIG. 3 is a perspective view illustrating an example of lyophilization by loading liquid reagent to a microfluidic device.

### DETAILED DESCRIPTION OF THE INVENTION

Hereinafter, the present invention will be described more fully with reference to the accompanying drawings, in which exemplary embodiments of the invention are shown.

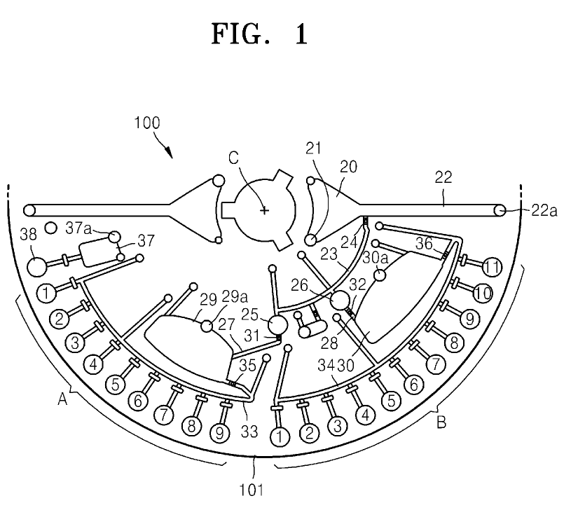

FIG. 1 is a diagram of a microfluidic device for a blood chemical analysis to which a method according to an embodiment of the present invention is applied. Referring to FIG. 1, the microfluidic device 100 includes a rotatable platform 101 (for example, a disc-type) and microfluidic structures constructed within the platform 101 for providing a space in which a fluid can be accommodated and a path through which the fluid can flow. The platform 101 can be rotated centering around the center C thereof. In the microfluidic structures within the platform 101, samples are moved, centrifuged, and mixed by the action of the centrifugal force generated by rotation of the platform 101.

The platform 101 can be formed of plastic materials such as acrylic and Polydimethylsiloxane (PDMS) in which the surfaces thereof is biologically non-activated. However, the materials of the platform 101 are not limited thereto and can be any materials having chemical and biological stability, optical transparency, and mechanical workability. The platform 101 can be formed of substrates having a number of layers. An engraved structure which corresponds to a chamber or a channel is formed on the surfaces of the substrates where the surfaces of the substrates face with each other and the substrates are combined to each other, thereby providing a space accompanied with a fluid and a path through which the fluid can flow within the platform 101. The substrates can be combined to each other by using various methods such as attaching with adhesives or both-sided adhesive tape, ultrasonic fusion, and laser bonding.

A series of the structures arranged within the platform 101 for blood test is described in more detail. Here, the area close to the center of the platform 101 is referred to as inside and the area distant from the center of the platform 101 is referred to as outside, for convenience. Firstly, a sample chamber 20 is disposed in the far inside of the platform 101. A certain amount of blood can be loaded to the sample chamber 20 from an exterior through a sample inlet 21. A centrifugal separator 22 is disposed outside of (i.e., radially outwards) the sample chamber 20 where the sample is separated into different components by the centrifugal force due to rotation of the platform 101. The centrifugal separator 22 includes a sediments collector 22a at the end part thereof, the sediments collector 22a accompanying with materials having large mass. The centrifugal separator 22 may be in the form of a channel. The dimension (i.e., width or depth) of the centrifugal separator 22 can be varied according to, for example, amounts of samples to be processed. A sample distribution channel 23 distributing collected serum into structures for subsequent process or reaction is connected to the centrifugal separator 22 through a valve 24. The sample distribution channel 23 may be disposed relatively radially inwards than reaction chamber, which will be discussed below. The sample distribution channel 23 is connected to the centrifugal separator 22 at its radially inward end, where it meets the sample chamber 20.

The valve 24 may be microfluidic valves in various forms. For example, a valve that is opened passively when a predetermined pressure is applied, or a valve that is operated actively upon the application of power or energy, for example from an external source. The valves may be a capillary valve or phase-change valve. In the current embodiment, a phase-change valve may be used. The phase-change valve is formed of a phase-change material which transforms its phase, for example from solid to liquid, upon application of energy (e.g., heat). The phase-change material is usually in a solid state at or around room temperature. A melted phase-change material is loaded into the sample distribution channel 23. When the phase-change material is solidified, the sample distribution channel 23 is blocked. When energy, which may be an electrical energy, optical energy, or in any other form, is applied to the phase-change material, the phase-change material is melted and, then solidified once the energy is removed. For example, the phase-change material may be wax. In an embodiment, the phase-change material may contain heating particles that are uniformly distributed in the phase-change material. The heating particles absorb electromagnetic energy and converting the electromagnetic energy into heat energy. Examples of the wax may include paraffin wax, microcrystalline wax, synthetic wax, or natural wax. The phase-change material may be gel or thermoplastic resin. Examples of the gel may include polyacrylamide, polyacrylates, polymethacrylates, or polyvinylamides. Also, examples of the thermoplastic resin may include cyclic olefin copolymer (COC), polymethylmethacrylate (PMMA), polycarbonate (PC), polystyrene (PS), polyoxymethylene (POM), perfluoralkoxy (PFA), polyvinylchloride (PVC), polypropylene (PP), polyethylene terephthalate (PET), polyetheretherketone (PEEK), polyamide (PA), polysulfone (PSU), and polyvinylidene fluoride (PVDF).

Sample chambers 25 and 26, which receive centrifugally separated sample, are disposed outward the sample distribution channel 23. The sample chambers 25 and 26 are respectively connected to dilution chambers 29 and 30 by channels 27 and 28. The channels 27 and 28 respectively include valves 31 and 32. A diluent is respectively loaded into the dilution chambers 29 and 30 through inlets 29a and 30a. In the dilution chambers 29 and 30, samples diluted to different ratios may be obtained. To obtain variously diluted samples, each of chambers 29 and 30 may contain different volumes of diluent from the other. Moreover, the amount of samples (serum) distributed to individual sample chambers 25 and 26 through the sample distribution channel 23 may vary. In other words, a different amount of serum can be respectively provided to the sample chambers 25 and 26. The valves 31 and 32 may be phase-change valves that are same to that of valve 24.

Reaction chamber groups A and B are respectively disposed outside (i.e., radially outward) the dilution chambers 29 and 30. The reaction chamber groups A and B may be formed of one reaction chamber, at the simplest, and may be formed of a plurality of reaction chambers, if needed. FIG. 1 illustrates the microfluidic device in which two clusters (Groups A and B) of reaction chambers are provided. In the reaction chamber group A, a plurality of the reaction chambers A1-A9 are arranged. The plurality of the reaction chambers A1-A9 are connected to the dilution chamber 29 through a common sample diluent distribution channel 33. In the reaction chamber group B, a plurality of the reaction chambers B1-B11 are arraigned. The plurality of the reaction chambers B1-B11 are connected to the dilution chamber 30 through a common sample diluent distribution channel 34. The capacities of the plurality of the reaction chambers A1-A9 and B1-B11 may be the same. However, the capacities of the individual chambers may be varied from each other. Valves 35 and 36 each selectively open the sample diluent distribution channels 33 and 34 and may be phase-change valves, as discussed above with respect to the valve 24.

In the reaction chambers A1-A9, reagents for employed for analyzing the sample (e.g., separated serum), for example, aspartate aminotransferase (AST), alanine aminotransferase (ALT), gamma glutamyl transferase (GGT), direct bilirubin (D-BIL), total bilirubin (T-BIL), creatin kinase (CK), Lactate Dehydrogenase (LDH), and amylase (AMY) can be respectively loaded. In the reaction chambers B1-B11, reagents for testing, for example, serum, Creatinine (CREA), Albumin (ALB), Total Protein (TP), calcium (Ca), Urea Nitrogen (BUN), Alkaline Phosphatase (ALP), glucose (GLU), total cholesterol (CHOL), triglycerides (TRIG), and Uric acid (UA) may be respectively loaded.

Meanwhile, an auxiliary dilution chamber 37 to which the sample is not provided from the sample distribution channel 23 can be disposed. The dilution chamber 37 is to obtain a reference value in reaction detection and a diluent can be stored in the dilution chamber 37. The diluent is loaded into the dilution chamber 37 through an inlet 37a. A chamber 38 for obtaining a detection reference value can be disposed outward the dilution chamber 37. Air bents for discharging air can be arranged in each chamber and channel, in case of need.

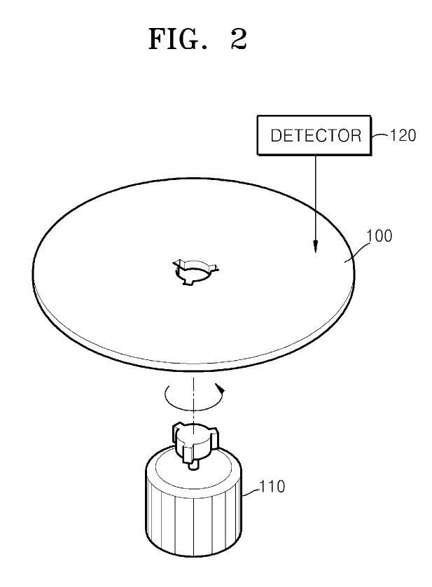

FIG. 2 is a schematic diagram of a blood analyzer using the microfluidic device 100. Referring to FIG. 2, a rotator unit 110 rotates the microfluidic device 100 for centrifusing the sample, providing separated serum to the sample chambers 25 and 26 (refer to FIG. 1) and the dilution chambers 29 and 30 (refer to FIG. 1), and providing diluted solutions from the dilution chambers 29 and 30 (refer to FIG. 1) to the reaction chambers A1-A9 and B1-B11 (refer to FIG. 1). Moreover, the rotator unit 110 stops the microfluidic device 100 at a predetermined location to face the reaction chambers A1-A9 and B1-B11 (refer to FIG. 1) with a detector 120. Although not illustrated in the rotator unit 110 of FIG. 2, the rotator unit 110 may include a motor drive for controlling an angular position of the microfluidic device 100. For example, the motor drive may use a step motor or a direct current motor. The detector 120 detects, for example, optical properties of materials to be detected such as fluorescence, light-emitting properties, and/or photoabsorption properties.

A blood analysis can be performed as follows. The numerical references are based on FIG. 1. Blood collected from a subject in need of blood analysis is loaded into the sample chamber 20 of the microfluidic device 100 in which a reagent is loaded and stored. A diluent may be loaded in advance and stored in the dilution chambers 29, 30, and/or 37 until the use of the device. Alternatively, the diluent may be loaded into the dilution chambers 29, 30, and/or 37 immediately prior to or during the operation.

Then, the microfluidic device 100 is installed to the blood analyzer of FIG. 2. The rotator unit 110 rotates the microfluidic device 100 to separate serum from blood and stops the microfluidic device 100. When the valve 24 (refer to FIG. 1) is opened, a predetermined amount of serum is provided to the sample chambers 25 and 26 (refer to FIG. 1) through the sample distribution channel 23 (refer to FIG. 1). Then, the valves 31 and 32 (refer to FIG. 1) are opened and serum is provided to the dilution chambers 29 and 30 (refer to FIG. 1) from the sample chambers 25 and 26 (refer to FIG. 1). The rotator unit 110 shakes the microfluidic device 100 right and left and serum and the diluent are mixed. Then, the valves 35 and 36 are opened and diluted serum is provided to the reaction chambers A1-A9 and B1-B11. The rotator unit 110 may shake the microfluidic device 100 right and left for few more times to mix the reagent and diluted serum.

Then, each of the reaction chambers A1-A9 and B1-B11 sequentially face with the detector 120 and whether materials to be detected exist in the reaction chambers A1-A9 and B1-B11 and, if exist, their amounts are measured.

In order to perform blood analysis described above, the microfluidic device may contain a certain amount of the reagent stored therein in advance. If such reagent-preloaded microfluidic device is used, a person who performs tests loads of a blood sample to the microfluidic device, followed by placing the sample-loaded microfluidic device into the blood analyzer so as to perform blood analysis. Hereinafter, a method of loading in advance and storing the reagent in the microfluidic device is described.

Firstly, a plurality of liquid reagents are loaded into a plurality of reaction chambers of the microfluidic device. The liquid reagent may have a higher concentration than the concentration needed for detecting materials to be analyzed in order to reduce the volume of the reagent loaded into the plurality of the reaction chambers.

A filler can be added to the liquid reagent. The filler allows the lyophilized reagent to have a porous structure so that when a sample diluent is loaded into the reaction chambers, the lyophilized reagent can be easily dissolved. For example, the filler may include, but is not limited to, bovine serum albumin (BSA), polyethylene glycol (PEG), dextran, mannitol, polyalcohol, myo-inositol, citric acid, ethylene diamine tetraacetic acid disodium salt (EDTA2Na), and polyoxyethylene glycol dodecyl ether (BRIJ-35). One or more fillers may be used for a respective reagent.

A surfactant can be added to the liquefied reagent. For example, the surfactant may include, but is not limited to, polyoxyethylene, lauryl ether, octoxynol, polyethylene alkyl alcohol, nonylphenol polyethylene glycol ether, ethylene oxide, ethoxylated tridecyl alcohol, polyoxyethylene nonylphenyl ether phosphate sodium salt, and sodium dodecyl sulfate. Here, one or more than two surfactants can be selected from among the above surfactants according to types of the reagents and can be added to the liquefied reagent.

As described above, the microfluidic device in which a fixed amount of the plurality of the liquefied reagents is loaded is put in a lyophilizer and a lyophilization process is performed according to a lyophilization program. The lyophilization program can be appropriately set according to the amount or types of the liquefied reagents.

The lyophilization denotes that moisture content contained in materials is frozen through a freezing process and then frozen moisture content is removed by drying. In general, sublimation in which frozen moisture content is directly changed into steam is used. In general, sublimation can be used in the lyophilization process. However, sublimation can be used only in some parts of the drying process, but not in the entire drying process. The pressure of the drying process can be reduced to below triple point of water (6 mbar or 4.6 Torr) for sublimation; however, the pressure may vary during the drying process. The temperature during the drying process can be changed and the temperature after freezing can be gradually increased.

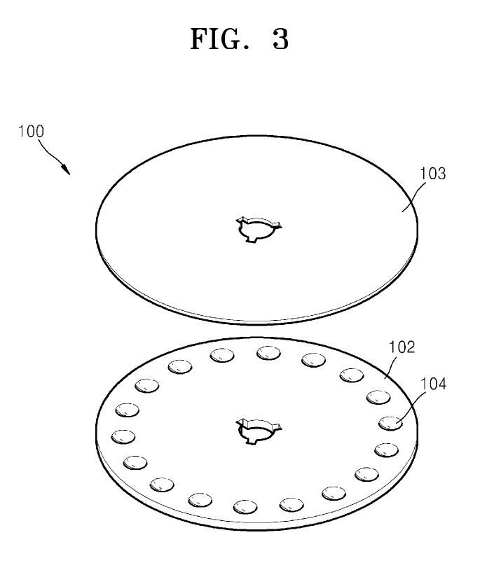

The microfluidic device having the structure as illustrated in FIG. 1 can be used. That is, although not illustrated in FIG. 1, the liquefied reagent can be loaded into the reaction chambers through inlets arranged in each reaction chamber. Moreover, the microfluidic device can be formed of a plurality of substrates. As illustrated in FIG. 3, the plurality of the liquefied reagents is loaded into a plurality of reaction chambers 104 arranged on a substrate 102 and the substrate 102 is put in a lyophilizer, thereby performing a lyophilization process. Then, a remaining substrate 103 may be combined to the substrate 102 by using methods such as bonding and fusing. Unlike FIG. 3, when the microfluidic device is formed of one reaction chamber, one liquefied reagent is loaded into the reaction chamber, thereby performing a lyophilization process and it is well known to one of ordinary skill in the art to which the invention pertains. Moreover, in FIG. 3, the reaction chambers 104 are only illustrated and it is well known to one of ordinary skill in the art that sample chambers and other microfluidic structures are omitted.

As described above, in the method of storing the reagent according to the embodiment of the present invention, the liquefied reagents are loaded into the reaction chambers of the microfluidic device so that the fixed amount of the reagents can be easily loaded. Moreover, since the microfluidic device is lyophilized at a time in the state that the liquefied reagents are loaded into the microfluidic device in advance, the microfluidic device used to analyze same testing items may be easily manufactured in large quantities.

For a performance test, the reagent is manufactured to have the double of the concentration of commercially available reagents for testing alanine aminotransferase (ALT), aspartate aminotransferase (AST), direct bilirubin (D-BIL), total bilirubin (T-BIL), gamma glutamyl transferase (GGT), Uric acid (UA), Albumin (ALB), amylase (AMY), creatin kinase (CK), Lactate Dehydrogenase (LDH), triglycerides (TRIG), total cholesterol (CHOL), glucose (GLU), and Urea Nitrogen (BUN) and then 50 μl of the reagent is loaded into each reaction chamber. Also, 50 μl of same samples is loaded into each of vials to verify the reagent. In the current embodiment, the fillers as shown in Table 1 is added.

TABLE 1

| | Items | Filler | Added Amount (g/L) |
|---|---|---|---|
| 1 | AST | DEXTRAN | 25 |
| 2 | ALT | EDTA 2NA | 18.8 |
| 3 | BUN | PEG 6000 | 50 |
| 4 | LDH | BSA | 25 |
| 5 | CK | PEG 6000 | 50 |
| 6 | GGT | PEG 6000 | 25 |
| 7 | AMY | DEXTRAN | 25 |
| 8 | CHOL | DEXTRAN | 25 |
| 9 | GLU | PEG 6000 | 25 |
| 10 | TRIG | PEG 6000 | 50 |
| 11 | UA | PEG 6000 | 25 |
| 12 | T-BIL | PEG 6000 | 75 |
| | | CITRIC ACID | 23.8 |
| 13 | D-BIL | PEG 6000 | 75 |
| | | CITRIC ACID | 23.8 |
| 14 | ALB | BRIJ-35 | 2 |

The plurality of the vials and the microfluidic device are put in a lyophilizer KM-12INT manufactured by C & H and a lyophilization process is performed according to the lyophilization program shown in table 2.

According to the lyophilization conditions illustrated in Table 2, the reagents freeze at an initial stage and then are dried by gradually increasing the temperature. In table 2, ‘NO’ denotes the order of the lyophilization program. In the lyophilization program, the pressure maintains under 25 millitorr within 20 minutes after a vacuum pump is operated and maintains under 25 millitorr until the final dried materials are taken out.

TABLE 2

| | Drying | Drying | |
|---|---|---|---|
| | Temperature | Time | |
| NO | (° C.) | (hr) | Description |
| 1 | −50 | 4 | Frozen |
| 2 | −40 | 1 | Heating |
| 3 | −40 | 3 | |
| 4 | −20 | 2 | Heating |
| 5 | −20 | 12 | |
| 6 | −10 | 2 | Heating |
| 7 | −10 | 4 | |
| 8 | 0 | 2 | Heating |
| 9 | 0 | 2 | |
| 10 | 10 | 1 | Heating |
| 11 | 10 | 13 | |
| 12 | 20 | 1 | Heating |
| 13 | 20 | 2 | |
| 14 | 30 | 1 | Heating |
| 15 | 30 | 2 | |

Performances of various testing reagents that are lyophilized in the same lyophilization condition are evaluated in terms of each item such as moisture content, solubility, initial light absorption, reaction completed light absorption, and linearity. Types of assay, normal ranges, wavelengths, and principles used for each item are shown in Table 3. Meanings of abbreviation used in Table 3 are as follows.

BCG: Bromocresol green

IFCC noPLP: International Federation of clinical chemistry, without pyridoxal phosphate without sample blank

BG7PNP: Ethylidene-4-nitrophenyl-α-D-maltoheptaoside

Urease GLDH: Urease.Glutamate dehydrogenase

COD-POD: Cholesterol oxidase. Peroxidase

DPD: 2.4-Dichlorophenyl diazonium-tetrafluoroborate

IFCC Glupa-C: International Federation of clinical chemistry, L-γ-Glutamyl-3-carboxy-4-nitroanilide

GOP-POD: Glucose oxidase. Peroxidase

Wroblewski P->L: Wroblewski. Pyruvate to lactare

LPL: Lipoprotein lipase

GPO: L-α-Glycerol phosphate oxidase

GK: Glycerokinase

Uricase-POD: Uricase-Peroxidase

TABLE 3

| Testing | Types Of | Wavelength | | |
|---|---|---|---|---|
| Items | Assay | (nm) | Normal Range | Principle |
| ALB | End Point | 620 | 3.7~5.2 (g/dl) | BCG |
| ALT | Kinetic | 340 | 5~35 (IU/L) | IFCC noPLP |
| AMY | Kinetic | 405 | 10~110 (IU/L) | BG7PNP |
| AST | Kinetic | 340 | 5~40 (IU/L) | IFCC noPLP |
| BUN | Kinetic | 340 | 8~20 (mg/dl) | Urease GLDH |
| CHOL | End Point | 500 | 130~250 | COD-POD |
| | | | (mg/dl) | |
| CK | Kinetic | 340 | M: 24~195 | UV Rate |
| | | | (mg/dl) | |
| | | | F: 24~170 | |
| | | | (mg/dl) | |
| D-BIL | End Point | 550 | 0.0~0.5 (mg/dl) | DPD |
| GGT | Kinetic | 405 | M: 0~50 | IFCC Glupa-C |
| | | | (mg/dl) | |
| | | | F: 0~30 (mg/dl) | |
| GLU | End Point | 500 | 70~110 (mg/dl) | GOP-POD |
| LD | Kinetic | 340 | 160~360 (IU/L) | Wroblewski P->L |
| T-BIL | End Point | 550 | 0.1~1.0 (mg/dl) | DPD |
| TRIG | End Point | 550 | M: 50~155 | LPL, GPO GX |
| | | | (mg/dl) | |
| | | | F: 40~115 | |
| | | | (mg/dl) | |
| UA | End Point | 550 | M: 3.9~6.9 | Uricase-POD |
| | | | (mg/dl) | |
| | | | F: 2.4~5.4 | |
| | | | (mg/dl) | |

### 1) Moisture Content

Moisture contents of the lyophilized reagents are examined to find out effect of lyophilization. As the result of the examination performed by a karl Fisher method, 14 testing reagents are well lyophilized as shown in Table 4 below.

TABLE 4

| | | Examination | Evaluation | |
|---|---|---|---|---|
| NO | Testing Item | Standard | Result | Result |
| 1 | AST | ≦10% | 7.1328 | Pass |
| 2 | ALT | ≦10% | 1.8489 | Pass |
| 3 | BUN | ≦10% | 2.6637 | Pass |
| 4 | LDH | ≦10% | 2.1236 | Pass |
| 5 | CK | ≦10% | 1.5815 | Pass |
| 6 | GGT | ≦10% | 2.0030 | Pass |
| 7 | AMY | ≦10% | 2.2868 | Pass |
| 8 | CHOL | ≦10% | 1.6669 | Pass |
| 9 | GLU | ≦10% | 2.3780 | Pass |
| 10 | TRIG | ≦10% | 1.9371 | Pass |
| 1 | UA | ≦10% | 4.8595 | Pass |
| 12 | T-BIL | ≦10% | 0.7308 | Pass |
| 13 | D-BIL | ≦10% | 1.4108 | Pass |
| 14 | ALB | ≦10% | 5.7982 | Pass |

### 2) Solubility

A diluent is loaded into the vials containing the lyophilized reagents and the vials are closed with a stopper. Then, the vials are shaken strongly and whether the lyophilized reagents are completely dissolved within seconds is examined. As shown in Table 5 below, excellent solubility is obtained for all 14 testing reagents.

TABLE 5

| NO | Testing Item | Examination Standard | Result |
|---|---|---|---|
| 1 | AST | Dissolved within 3 seconds by | Pass |
| | | strongly shaking | |
| 2 | ALT | Dissolved within 3 seconds by | Pass |
| | | strongly shaking | |
| 3 | BUN | Dissolved within 3 seconds by | Pass |
| | | strongly shaking | |
| 4 | LDH | Dissolved within 3 seconds by | Pass |
| | | strongly shaking | |
| 5 | CK | Dissolved within 3 seconds by | Pass |
| | | strongly shaking | |
| 6 | GGT | Dissolved within 3 seconds by | Pass |
| | | strongly shaking | |
| 7 | AMY | Dissolved within 3 seconds by | Pass |
| | | strongly shaking | |
| 8 | CHOL | Dissolved within 3 seconds by | Pass |
| | | strongly shaking | |
| 9 | GLU | Dissolved within 3 seconds by | Pass |
| | | strongly shaking | |
| 10 | TRIG | Dissolved within 3 seconds by | Pass |
| | | strongly shaking | |
| 11 | UA | Dissolved within 3 seconds by | Pass |
| | | strongly shaking | |
| 12 | T-BIL | Dissolved within 3 seconds by | Pass |
| | | strongly shaking | |
| 13 | D-BIL | Dissolved within 3 seconds by | Pass |
| | | strongly shaking | |
| 14 | ALB | Dissolved within 3 seconds by | Pass |
| | | strongly shaking | |

### 3) Initial Light Absorption

Device for measuring: Hitachi-U3010 spectrophotometer

Number of samples: 3 reagents for each testing item

Only a diluent is mixed to the lyophilized reagents and initial light absorption is measured by time-scanning for five minutes. As a result, excellent results can be obtained as illustrated in Table 6 below. The initial light absorption test is to evaluate a light absorption value of the reagent itself before inserting serum into the reagent and to identify any problem during the lyophilization process. The examination standard values denotes values using the reagents before inserting new optimized additives to the reagents during the lyophilization process and the evaluation result denotes that the lyophilized reagents are used after optimizing the additives.

TABLE 6

Initial light absorption (abs)

| | Testing | Examination | Evaluation | |
|---|---|---|---|---|
| NO | Item | Standard | Result | Result |
| 1 | AST | 1.3-1.5 | 1.409-1.415 | pass |
| 2 | ALT | 1.3-1.5 | 1.437-1.443 | pass |
| 3 | GGT | ≦0.8 | 0.672-0.680 | pass |
| 4 | T-BIL | ≦0.05 | 0.010-0.013 | pass |
| 5 | D-BIL | ≦0.05 | 0.007-0.008 | pass |
| 6 | GLU | ≦0.05 | 0.023-0.024 | pass |
| 7 | TRIG | ≦0.1 | 0.071-0.078 | pass |
| 8 | UA | ≦0.02 | 0.008 | pass |
| 9 | LDH | 1.6-1.8 | 1.764-1.773 | pass |
| 10 | CK | ≦0.3 | 0.154-0.159 | pass |
| 1 | AMY | ≦0.1 | 0.023-0.025 | pass |
| 12 | BUN | 1.6-1.8 | 1.561-1.581 | Pass |
| 13 | CHOL | ≦0.05 | 0.010-0.011 | Pass |
| 14 | ALB | ≦0.210 | 0.203-0.207 | Pass |

### 4) Reaction Completion

With regard to the item “end-point,” in which a final value after a predetermined time of the reaction is used, if the reaction continuously proceeds without completing after five minutes, reproducibility of the test result is affected. Accordingly, in order to identify whether the reaction is completed within 5 minutes, the light absorption is time-scanned for five minutes and changes of the light absorption are observed at fourth and fifth minutes. As a result, it is identified that the reaction is saturated within the standard time as shown in Table 7. Regarding the testing item “Kinetic,” in which reaction change rates per minute is measured, the reaction completion test is not significant and thus is not measured. Here, normal standard serum, Muli-sera normal-Lot No. 19236A, and abnormal standard serum, Muli-sera abnormal-Lot No. 19239A, provided by Linear Chemicals are used and a Hitachi-U3010 spectrophotometer is used to measure these serums.

TABLE 7

| | | Reaction Completion (abs) | | | |
|---|---|---|---|---|---|
| | | | Evaluation Result | | |
| | | | Normal | | |
| | Testing | Examination | standard | abnormal | |
| NO | Item | Standard | serum | standard serum | Result |
| 1 | CHOL | ≦0.02 | 0.003 | 0.004 | Pass |
| 2 | GLU | ≦0.02 | 0.004 | 0.001 | Pass |
| 3 | TRIG | ≦0.02 | 0.006 | 0.003 | Pass |
| 4 | UA | ≦0.02 | 0.001 | 0.001 | Pass |
| 5 | T-BIL | ≦0.02 | 0.001 | 0.001 | Pass |
| 6 | D-BIL | ≦0.02 | 0.001 | 0.002 | Pass |
| 7 | ALB | ≦0.02 | 0.003 | 0.003 | Pass |

### 5) Linearity

Device for measuring: Hitachi-U3010 spectrophotometer

Standard serum used: normal standard serum, Muli-sera normal-Lot No. 19236A, and abnormal standard serum, Muli-sera abnormal-Lot No. 19239A provided by Linear Chemicals.

Five concentration samples in which above two serums are mixed in the ratio of 4:0, 3:1, 2:2, 1:3, and 0:4 are measured four times for each sample and a dynamic range of the lyophilized reagents is measured. Then, linearity of the measured result is examined. As shown in Table 8, excellent linearity can be obtained.

An excellent linearity in a given concentration range indicates an enhancement of accuracy of the concentration estimated by using only changes of the light absorption.

TABLE 8

linearity (R²)

| | Testing | Examination | Evaluation | |
|---|---|---|---|---|
| NO | Item | Standard | Result | Result |
| 1 | AST | ≧0.95 | 0.9997 | Pass |
| 2 | ALT | ≧0.95 | 0.9997 | Pass |
| 3 | GGT | ≧0.95 | 0.9970 | Pass |
| 4 | T-BIL | ≧0.95 | 0.9985 | Pass |
| 5 | D-BIL | ≧0.95 | 0.9994 | Pass |
| 6 | GLU | ≧0.95 | 0.9985 | Pass |
| 7 | TRIG | ≧0.95 | 0.9943 | Pass |
| 8 | UAL | ≧0.95 | 0.9941 | Pass |
| 9 | LDH | ≧0.95 | 0.9984 | Pass |
| 10 | CK | ≧0.95 | 0.9993 | Pass |
| 1 | AMY | ≧0.95 | 0.9874 | Pass |
| 12 | BUN | ≧0.95 | 0.9958 | Pass |
| 13 | CHOL | ≧0.95 | 0.9998 | Pass |
| 14 | ALB | ≧0.95 | 0.9990 | Pass |

### 6) Reproducibility

20 samples for each of 14 testing items are examined by using normal standard serum, Muli-sera normal-Lot No. 19236A, and abnormal standard serum, Muli-sera abnormal-Lot No. 19239A provided by Linear Chemicals. As a result, excellent reproducibility within 5% of a coefficient of variation can be obtained as shown in Table 9. Here, autochemistry analyzer AMS-19 is used.

TABLE 9

| | | Reproducibility (CV %) | | | |
|---|---|---|---|---|---|
| | | | Evaluation Result | | |
| | | | normal | | |
| | Testing | Examination | standard | abnormal | |
| NO | Item | Standard | serum | standard serum | Result |
| 1 | AST | ≦5 | 4.51 | 2.83 | Pass |
| 2 | ALT | ≦5 | 4.98 | 3.70 | Pass |
| 3 | BUN | ≦5 | 3.16 | 4.72 | Pass |
| 4 | LDH | ≦5 | 3.65 | 3.24 | Pass |
| 5 | CK | ≦5 | 3.90 | 3.99 | Pass |
| 6 | GGT | ≦5 | 3.76 | 3.24 | Pass |
| 7 | AMY | ≦5 | 4.05 | 4.41 | Pass |
| 8 | CHOL | ≦5 | 2.09 | 2.19 | Pass |
| 9 | GLU | ≦5 | 2.78 | 1.97 | Pass |
| 10 | TRIG | ≦5 | 2.53 | 1.60 | Pass |
| 1 | UA | ≦5 | 0.70 | 1.52 | Pass |
| 12 | T-BIL | ≦5 | 3.71 | 3.25 | pass |
| 13 | D-BIL | ≦5 | 2.53 | 4.55 | pass |
| 14 | ALB | ≦5 | 1.12 | 1.31 | pass |

Consequently, from the examination result, the plurality of the liquid reagents is loaded into the microfluidic device and then the microfluidic device can be lyophilized. According to such a method of storing of the liquid reagent, efforts to forming lyophilized reagent beads having the low volume (accurately controlled) and the difficulty of loading the reagent beads in a solid state into the disk-type microfluidic device are avoidable. Moreover, existing liquid reagents are directly applied to the automated disk-type microfluidic device so that excellent economical efficiency and compatibility can be realized.

In the above description, the microfluidic device including one sample chamber connected to two serum chambers has been described; however, this is only an example.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of storing a reagent in a microfluidic device, wherein the microfluidic device is provided with a reaction chamber and fluid paths, the method comprising:
loading the reagent into the reaction chamber; and
lyophilizing the reagent in the state that the reagent is loaded into the microfluidic device.

2. The method of claim 1, wherein the reagent is loaded into the reaction chamber, in a liquid form.

3. The method of claim 2, wherein the liquid reagent has a concentration higher than a concentration that is used in the assay.

4. The method of claim 1, wherein the lyophilizing comprises a freezing process and a drying process, and the drying process uses sublimation in at least some portions of the drying process.

5. The method of claim 1, wherein the microfluidic device comprises at least two reaction chambers and the loading the reagent comprises loading a plurality of the reagents that are different from each other into the reaction chambers.

6. The method of claim 5, wherein the plurality of the reagents comprise at least two selected from reagents used for testing aspartate aminotransferase (AST), alanine aminotransferase (ALT), gamma glutamyl transferase (GGT), direct bilirubin (D-BIL), total bilirubin (T-BIL), creatin kinase (CK), Lactate Dehydrogenase (LDH), amylase (AMY), Creatinine (CREA), Albumin (ALB), Total Protein (TP), calcium (Ca), Urea Nitrogen (BUN), Alkaline Phosphatase (ALP), glucose (GLU), total cholesterol (CHOL), triglycerides (TRIG), and Uric acid (UA).

7. The method of claim 1, further comprising adding a filler into the reagent before lyophilizing the reagent.

8. The method of claim 7, wherein the filler comprises at least one of bovine serum albumin (BSA), polyethylene glycol (PEG), dextran, mannitol, polyalcohol, myo-inositol, and citric acid.

9. The method of claim 1, further comprising adding a surfactant into the reagent before lyophilizing the reagent.

10. The method of claim 9, wherein the surfactant comprises at least one selected from the group consisting of polyoxyethylene, lauryl ether, octoxynol, polyethylene alkyl alcohol, nonylphenol polyethylene glycol ether; ethylene oxid, ethoxylated tridecyl alcohol, polyoxyethylene nonylphenyl ether phosphate sodium salt, and sodium dodecyl sulfate.

11. A method for producing a microfluidic device suitable for an assay of a biological sample, the microfluidic device being provided with a reaction chamber and fluid paths and containing a reagent for performing the assay in the reaction chamber, the method comprising:
loading the reagent in liquid form into the reaction chamber of the device; and
lyophilizing the reagent in the state that the reagent is loaded into the microfluidic device.
